(19) United States
(12) Patent Application Publication
(10) Pub. No.: US 2024/0424183 A1
(43) Pub. Date: Dec. 26, 2024

(54) DEVICES, SYSTEMS AND METHODS FOR THE BROAD-SPECTRUM REDUCTION OF PRO-INFLAMMATORY CYTOKINES IN BLOOD

(71) Applicant: Sigyn Therapeutics, Inc., San Diego, CA (US)

(72) Inventors: Craig P. Roberts, Oceanside, CA (US); James A. Joyce, San Diego, CA (US)

(21) Appl. No.: 18/827,495

(22) Filed: Sep. 6, 2024

Related U.S. Application Data
(63) Continuation of application No. 16/943,436, filed on Jul. 30, 2020.
(60) Provisional application No. 62/881,740, filed on Aug. 1, 2019.

(51) Int. Cl. A61M 1/34; A61M 1/36; B01D 63/02

(52) U.S. Cl. CPC A61M 1/3496; A61M 1/3653; B01D 63/034; A61M 2202/0415; A61M 2202/049; A61M 2205/7509; A61M 2205/7518

(57) ABSTRACT

Devices, systems and methods for the broad-spectrum reduction of pro-inflammatory cytokines in blood. The pro-inflammatory cytokines can be freely circulating in the blood as well as cytokines that are transported within or bound to the surface of particles collectively referred to as CytoVesicles while simultaneously adsorbing toxins and pathogens from blood and blood plasma. A plasma separation column with binding, capture and adsorbent components optimize the removal of cytokines and CytoVesicles from blood while minimizing the removal of essential blood elements. Adsorbent components are incorporated within the extra-lumen space, outside of the plasma fiber walls and within the outer shell of the column and can include activated carbon, ion exchange resins and non-ionic exchange resins. The resulting devices, systems and methods alleviate the symptoms or severity of a wide range of disease conditions associated with an abnormal production or dysregulation of pro-inflammatory cytokines.

### RELATED APPLICATIONS

This application is a continuation of and claims priority to U.S. application Ser. No. 16/943,436, filed on Jul. 30, 2020, which claims priority to U.S. Provisional Application No. 62/881,740 entitled DEVICES, SYSTEMS AND METHODS FOR BROAD-SPECTRUM REDUCTION OF PRO-INFLAMMATORY CYTOKINES IN BLOOD, filed on Aug. 1, 2019. The entire disclosures of the related applications set forth in this section is hereby incorporated by reference.

### FIELD OF THE INVENTION

The present invention relates to devices, systems and methods to reduce the presence of inflammatory particles from blood and blood plasma.

### BACKGROUND

Human cytokine production plays a central role to stimulate and regulate the innate and adaptive immune response to inflammation, trauma and infection. Cytokines represent a family of more than 100 different immune response agents, which can exist in peptide, and protein forms. Included within the cytokine family are chemokines, interferons, interleukins, lymphokines and tumor necrosis factor. Cytokines may circulate freely in the bloodstream or be transported within or bound to the surface larger particles known as Cytovesicles.

An abnormal production or dysregulation of cytokines has been associated with a wide range of diseases. The excessive chronic production of pro-inflammatory cytokines has been linked to atherosclerosis, cancer, cystic fibrosis, rheumatoid arthritis and neurological disorders, including Alzheimer's disease.

An excessive acute production or dysregulation of pro-inflammatory cytokines may cause or con-tribute to life-threatening conditions, which include but are not limited to cytokine storm syndrome (CSS), virus induced cytokine storm, bacteria induced cytokine storm, acute respiratory distress syndrome (ARDS), cytokine release syndrome (CRS), graft-versus-host disease (GVHD), sepsis, systemic inflammatory response syndrome (SIRS), hepatic encephalopathy, acute kidney injury (AKI) and severe pneumonia. Beyond cytokine and CytoVesicles, circulating toxins and pathogens may contribute to the initiation or progression of acute inflammatory conditions.

At present, there is a significant unmet medical need for anti-cytokine therapies to address disease conditions associated with the abnormal or dysregulated production of pro-inflammatory cytokines. It should be noted that this Background is not intended to be an aid in determining the scope of the claimed subject matter nor be viewed as limiting the claimed subject matter to implementations that solve any or all of the disadvantages or problems presented above. The discussion of any technology, documents, or references in this Background section should not be interpreted as an admission that the material described is prior art to any of the subject matter claimed herein.

It is understood that various configurations of the subject technology will become apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### SUMMARY OF INVENTION

Disclosed herein are devices, systems and methods to reduce the presence of inflammatory particles from blood and blood plasma. In one implementation, an extracorporeal system for the removal of inflammatory agents from blood is provided. Advantageously, the system includes a housing and a hollow fiber filter disposed within the housing. The filter may include a plurality of pores sized and dimensioned to permit passage of inflammatory agents having a diameter between about 0.5 nanometers and 6000 nanometers. The system further includes an adsorption component positioned inside the housing and outside the hollow fiber in an extra-lumen space.

In one aspect, the adsorption component is activated carbon, non-ionic exchange resin or ion exchange resin.

In another aspect, the inflammatory agents can include circulating cytokines, proteins with surface-bound cytokines, CytoVesicles with encapsulated cytokine cargos, CytoVesicles with surface bound cytokines, pathogens, endotoxins, and/or exotoxins.

A plurality of pores permit the inflammatory agents with diameters less than 0.60 microns to pass through the fiber walls and interact with the adsorption components. Additionally, the plurality of pores are sized and dimensioned to prevent a blood agent having a diameter greater than 0.60 microns to pass through the fiber walls and interact with adsorption components in the extra-lumen space, thereby preserving the integrity of the blood agents during filtration.

With regard to the adsorbents, it will be appreciated that the activated carbon can be a coated or uncoated coconut shell granule. Alternatively, the activated carbon can be a synthetic charcoal. Optionally, the adsorbent is at least one ion exchange resin or non-ionic exchange resin. The non-ionic exchange resin may be a non-ionic aliphatic ester resin, non-ionic polystyrene divinyl benzene resin, or other non-biologic adsorptive resins.

In yet another aspect, the at least one of said non-ionic aliphatic ester resins has an average surface area of approximately 500 m2/g, an average pore size of approximately 300-600 angstroms, and a mean particle diameter of 560 microns. In another aspect, the non-ionic polystyrene divinyl benzene resins has an average surface area of approximately 700 m2/g, and an average pore size of 300 angstroms, and a mean particle diameter from approximately 35 microns to approximately 120 microns. In still another aspect, the non-ionic polystyrene divinyl benzene resins has an average surface area of approximately 600 m2/g, an average pore size of 100-400 Angstroms, and a mean particle diameter from approximately 300 microns to approximately 500 microns.

Advantageously, the activated carbon has a pore size distribution of a Micropore region of less than 100 Angstroms, a Mesopore region of between 100 and 1,000 Angstroms, and a Macropore region of greater than 1,000 Angstroms.

In another aspect of the invention, a method for treating a disease or disorder in an individual in need thereof is disclosed. The method includes providing an extracorporeal adsorptive toxin removal device. The toxin removal device has a housing; a hollow fiber plasma filter having a number of pores sized between 200-6000 Angstrom; and an adsorbent positioned inside the housing and outside the fiber in the extra lumen space. Plasma is filtered through the adsorptive toxin removal device to cause an inflammatory causing agent with a diameter less than 0.60 microns to pass through the pores. The method also includes contacting the inflammatory causing agent with the absorbent; wherein the inflammatory causing agent binds to the adsorbent; and capturing the inflammatory causing agent in the adsorbent. The capture of the inflammatory causing agent may prevent the agent from reentering circulation.

The inflammatory agent may be a pro-inflammatory cytokine such as IL-1, TNF-a, IL-6, IL-11, IL-8, G-CSF, and GM-CSF, IL-3, IL-5, IL-7, IL-9, and transforming growth factor-b (TGF-b). Optionally, the inflammatory agent is an agent which contributes to cellular inflammation such as IL-1, IL-2, IL-3, IL-4, IL-6, IL-7, IL-9, IL-10, IL-12, IL-13 interferons (IFNs), IFN-g inducing factor (IGIF), TGF-b, and TNF-a and -b.

The removal of inflammatory agents can treat a disease or disorder such as cytokine storm syndrome (CSS), virus induced cytokine storm, bacteria induced cytokine storm, acute respiratory distress syndrome (ARDS), cytokine release syndrome (CRS), graft-versus-host disease (GVHD), sepsis, systemic inflammatory response syndrome (SIRS), hepatic encephalopathy, acute kidney injury (AKI) and pneumonia.

In yet another implementation, the inflammatory agent is a biologic toxin. The toxin is a bacterial endotoxin or exotoxin. The bacteria may be gram positive or gram negative.

In another aspect, a method of simultaneously removing circulating cytokines, CytoVesicles, cytokine aggregates, and endotoxins from the blood of an individual is disclosed. The method may include: accessing an extracorporeal line to an individual's circulatory system with a catheter; providing the extracorporeal system as disclosed herein, wherein the system has an inlet port and an outlet port; connecting the line to the inlet port; attaching a second extracorporeal line to the outlet port; controlling the flow of blood through the extracorporeal system with a pump; filtering the blood through the hollow fibers of the extracorporeal system; wherein filtering causes the circulating cytokines, CytoVesicles, cytokine aggregates, and endotoxins to pass through the pores and into the extra-lumen space; and contacting the filtered circulating cytokines, CytoVesicles, cytokine aggregates, and endotoxins with the adsorbent; wherein the adsorbent captures the cytokines, CytoVesicles, cytokine aggregates, and endotoxins.

It is understood that various configurations of the subject technology will become apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

Various embodiments are discussed in detail in conjunction with the Figures described below, with an emphasis on highlighting the advantageous features. These embodiments are for illustrative purposes only and any scale that may be illustrated therein does not limit the scope of the technology disclosed. These drawings include the following figures, in which like numerals indicate like parts.

FIG. 1 is a graphic representation of the cellular response to inflammation.

FIG. 2 is a perspective view of an embodiment of an extracorporeal device according to an aspect of the invention, wherein the housing is transparent to illustrate the hollow fibers and absorbent components.

FIG. 3 is a schematic representation of the use of an extracorporeal device for the filtration of plasma and capture of inflammatory causing agents.

### DETAILED DESCRIPTION

The following description and examples illustrate some exemplary implementations, embodiments, and arrangements of the disclosed invention in detail. Those of skill in the art will recognize that there are numerous variations and modifications of this invention that are encompassed by its scope. Accordingly, the description of a certain example embodiment should not be deemed to limit the scope of the present invention.

Implementations of the technology described herein are directed generally to cytokine capture in the blood via an extracorporeal device. To facilitate an understanding of the various embodiments described herein, a number of terms are defined below.

### General Interpretive Principles for the Present Disclosure

Various aspects of the novel systems, apparatuses, and methods are described more fully hereinafter with reference to the accompanying drawings. The teachings disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the disclosure. For example, a system or an apparatus may be implemented, or a method may be practiced using any one or more of the aspects set forth herein. In addition, the scope of the disclosure is intended to cover such a system, apparatus or method which is practiced using other structure, functionality, or structure and functionality in addition to or other than the various aspects of the disclosure set forth herein. It should be understood that any aspect disclosed herein may be set forth in one or more elements of a claim. Although some benefits and advantages of the preferred aspects are mentioned, the scope of the disclosure is not intended to be limited to particular benefits, uses, or objectives. The detailed description and drawings are merely illustrative of the disclosure rather than limiting, the scope of the disclosure being defined by the appended claims and equivalents thereof.

With respect to the use of plural vs. singular terms herein, those having skill in the art can translate from the plural to the singular and/or from the singular to the plural as is appropriate to the context and/or application. The various singular/plural permutations may be expressly set forth herein for sake of clarity.

When describing an absolute value of a characteristic or property of a thing or act described herein, the terms “substantial,” “substantially,” “essentially,” “approximately,” and/or other terms or phrases of degree may be used without the specific recitation of a numerical range. When applied to a characteristic or property of a thing or act described herein, these terms refer to a range of the characteristic or property that is consistent with providing a desired function associated with that characteristic or property.

In those cases where a single numerical value is given for a characteristic or property, it is intended to be interpreted as at least covering deviations of that value within one significant digit of the numerical value given.

If a numerical value or range of numerical values is provided to define a characteristic or property of a thing or act described herein, whether or not the value or range is qualified with a term of degree, a specific method of measuring the characteristic or property may be defined herein as well. In the event no specific method of measuring the characteristic or property is defined herein, and there are different generally accepted methods of measurement for the characteristic or property, then the measurement method should be interpreted as the method of measurement that would most likely be adopted by one of ordinary skill in the art given the description and context of the characteristic or property. In the further event there is more than one method of measurement that is equally likely to be adopted by one of ordinary skill in the art to measure the characteristic or property, the value or range of values should be interpreted as being met regardless of which method of measurement is chosen.

It will be understood by those within the art that terms used herein, and especially in the appended claims (e.g., bodies of the appended claims) are intended as “open” terms unless specifically indicated otherwise (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

In those instances where a convention analogous to “at least one of A, B, and C” is used, such a construction would include systems that have A alone, B alone, C alone, A and B together without C, A and C together without B, B and C together without A, as well as A, B, and C together. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include A without B, B without A, as well as A and B together.”

Various modifications to the implementations described in this disclosure can be readily apparent to those skilled in the art, and generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “exemplary” is used exclusively herein to mean “serving as an example, instance, or illustration.” Any implementation described herein as “exemplary” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

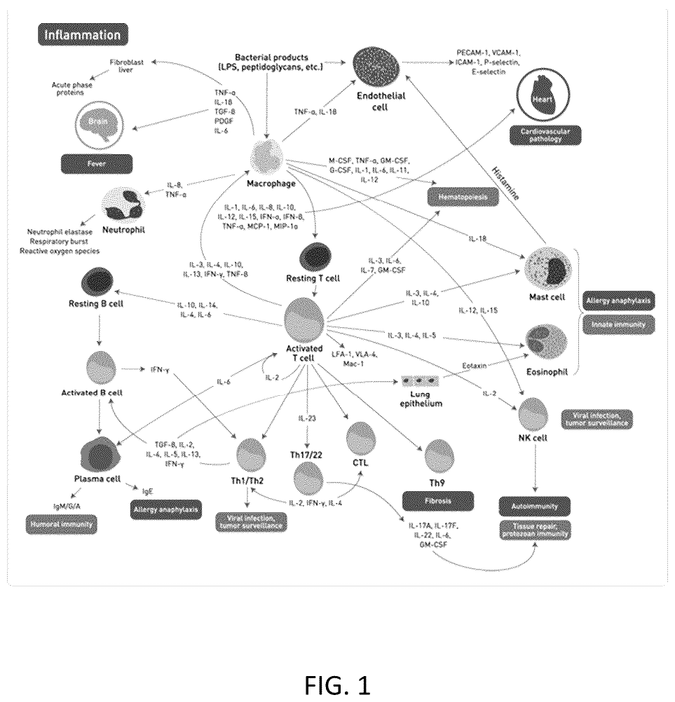

Inflammation is a natural response of the human immune system to promote recovery from injury or defend against infection. An important element of the host immune system is the release of molecular messengers known as cytokines, which regulate the response to disease, injury or infection, as well as mediate normal cellular processes in the body. As illustrated in FIG. 1, the production of pro-inflammatory cytokines is a prerequisite for initiating the anti-infectious process, whereas their exacerbated production during severe inflammation may contribute to significant negative consequences.

Indeed, numerous infectious and non-infectious disease conditions have been associated with an exuberant immune response that results in the excessive production of inflammatory cytokines, which among many terms, has been described as hypercytokinemia or Cytokine Storm Syndrome (Cytokine Storm). At present, the treatment of conditions that are precipitated by excess cytokine production is primarily limited to supportive care as direct acting therapeutic drug candidates have failed to demonstrate efficacy in controlled human studies. As a result, there is a significant unmet global need for a therapy that can safely modulate excessive inflammatory cytokine production.

In chronic disease conditions, the excess production of inflammatory cytokines has been associated with cancer, cardiovascular disease and numerous auto-immune conditions, including arthritis.

In acute inflammatory disorders, unmet therapeutic needs include, but are not limited to the treatment of virus induced cytokine storm that underlies a broad-spectrum of viral pathogens (including COVID-19), bacteria induced cytokine storm, acute respiratory distress syndrome (ARDS) and acute forms of liver failure, including hepatic encephalopathy. Excessive cytokine production may also result from trauma, severe burns, acute pancreatitis, cancer immunotherapies, cancer cachexia, acute kidney injury and severe pneumonia.

Sepsis, defined as a life-threatening organ dysfunction caused by a dysregulated host response, is perhaps the most prevalent condition resulting from the excess production of inflammatory cytokines. In January of 2020, a report entitled; “Global, Regional, and National Sepsis Incidence and Mortality, 1990-2017: Analysis for the Global Burden of Disease Study,” reported 48.9 million cases of sepsis and 11 million sepsis related deaths in 2017. In that same year, an estimated 20.3 million sepsis cases and 2.9 million deaths were among children younger than 5-years old.

To date, more than 100 human clinical studies have been conducted to evaluate the safety and benefit of candidate drugs to treat sepsis. With one brief exception (Xigris, Eli Lilly), none of these studies have resulted in a market approved therapy.

As the treatment of sepsis and other life-threatening inflammatory conditions remains elusive for therapeutic drugs, an increased understanding of complex mechanisms that underlie sepsis and other inflammatory conditions has established a basis for therapeutic strategies that modulate a broad-spectrum of inflammatory factors. As a result, an increased focus has been directed toward extracorporeal blood purification, with an emphasis on devices to improve immune homeostasis through the depletion of cytokines and other inflammatory mediators in the bloodstream.

Among previous extracorporeal blood purification strategies, have been systems that incorporated adsorbent components to address life-threatening inflammatory conditions. Among these strategies are the coupled plasma filtration adsorption (CPFA) system from Bellco Industries and the Hemolife IMPACT (Intermittent Modular Plasma Adsorption of Cytokines and Toxins) system. Beyond their expense, these systems proved complex to administer as they each required a series of three cartridges and additional pumps. Both the CPFA and IMPACT therapies circulated patient blood into a plasma filter as a means to separate plasma from cellular blood components. The isolated plasma was then redirected to a cartridge that contained an adsorbent component that targeted inflammatory cytokines. As plasma exited the adsorbent cartridge, it was then pumped to circulate through a third filtration device prior to being re-infused back into the patient's circulatory system.

Recent advances in extracorporeal blood purification have led to the design of single “blood-in-blood-out” cartridges that can be deployed within the established infrastructure of hemodialysis and continuous renal replacement therapy (CRRT) machines that are located in hospitals and clinics worldwide. Unlike the CPFA and IMPACT system, additional cartridges and pumps are not required.

The most prevalent “blood-in-blood-out” technologies are the Toraymyxin device developed by Toray Industries and the CytoSorb device developed by CytoSorbents Corporation. Both devices are market cleared in more than 40 countries. Toraymyxin has been safely administered to more than 150,000 patients and is the subject of more than 200 publications. CytoSorb has been safely administered to more than 80,000 patients.

Toraymyxin incorporates an antibiotic with a specificity to bind circulating endotoxin, which is a potent activator of inflammatory cytokines induced by bacterial infections. However, Toraymyxin does not address cytokines and other inflammatory targets. Conversely, the CytoSorb device incorporates an adsorbent with pores that allow for the depletion of circulating cytokines below 5 nanometers in diameter, but does not address endotoxin or inflammatory particles larger than 5 nanometers in diameter.

The devices, systems and methods described herein provide for the simultaneous depletion of inflammatory particles and activators from the bloodstream. Such targets may include, but are not limited to cytokines and endotoxin that circulate freely in the blood as well as inflammatory targets that have not been the focus of previous drug and device therapies. Included among these targets are cytokine aggregates and CytoVesicles that transport inflammatory cytokines and other mediators of inflammation.

Sepsis and many other inflammatory conditions are precipitated by an excess production of inflammatory cytokines, which can form into aggregates, be promoted through bacterial endotoxin, and/or transported as cargo within or on the surface of CytoVesicles. Similarly, most life-threatening inflammatory conditions are not addressed with a drug or device that has been proven safe and efficacious in controlled human studies. As a result, there is a significant unmet global need for a therapy to treat a broad-spectrum of life-threatening inflammatory conditions.

As the hallmark of inflammatory disease and conditions is the excess production inflammatory cytokines, previous therapeutic candidates have often focused on specific cytokine targets and did not take into consideration the considerable breadth of other particles that act in concert with cytokines to promote life-threatening inflammatory conditions.

Described herein is an extracorporeal technology that establishes a therapeutic modality to simultaneously address a broad-spectrum of particles that promote inflammatory diseases and conditions.

### Definitions

As used herein, cytokines are defined to refer to a family of more than 100 different immunomodulation agents, which can exist in both peptide and protein forms. Included within the cytokine family are chemokines, interferons, interleukins, lymphokines and tumor necrosis factor.

As used herein, an endotoxin is defined as any of a toxin present inside a bacterial cell and released when the cell disintegrates. An endotoxin is a potent driver of inflammatory cytokine production that can result from a broad-spectrum of bacterial infections, including drug-resistant species.

A cytokine aggregate is defined as a formation (whose diameter exceeds 5 nanometers) comprised of two or more cytokines that have bound together in circulation.

As used herein, CytoVesicles refer to particles (whose diameter exceeds 5 nanometers) which transport cytokines and other inflammatory cargos in the bloodstream. CytoVesicles are inclusive of microparticles (MPs) and microvesicles (MVs) classified as Extracellular Vesicles (EVs) with cytokines bound to their surface as well as EVs that transport cytokines as encapsulated cargo. CytoVesicle populations may also include platelet-derived MVs, endothelial-derived MVs and leukocyte-derived MVs, which are often prevalent in the blood of those suffering from acute and chronic inflammatory disorders.

The term “inflammatory particle” as used herein refers to inflammatory cytokines, endotoxin, cytokine aggregates and CytoVesicles that circulate in biological fluids, including blood and blood plasma.

Among current cytokine-targeting therapies are antibody drug agents that align with single cytokine targets and an extracorporeal device known as CytoSorb, which reduces the presence of cytokines that circulate freely in blood. However, neither therapeutic strategy incorporates a mechanism to address biologically active cytokines that are transported within or bound to the surface of particles collectively defined as CytoVesicles. Examples of CytoVesicles include but are not limited to microparticles (MPs) and microvesicles (MPs) classified as Extracellular Vesicles (EVs) with cytokines bound to their surface as well as EVs that transport cytokines as encapsulated cargo. CytoVesicle populations may also include platelet-derived MVs, endothelial-derived MVs and leukocyte-derived MVs, which are prevalent in the blood of those suffering from acute and chronic inflammatory disorders.

While cytokine production is a vital component of the normal immune response to inflammation, trauma and infection; the overproduction or dysregulation of pro-inflammatory cytokines may contribute to the pathogenesis of a wide range of disease conditions. The devices, systems and methods disclosed herein support the broad-spectrum reduction of freely circulating cytokines and CytoVesicles from blood. These include, but are not limited to, the following cytokines: IL-1, TNF-alpha, IL-6, IL-11, IL-8 and other chemokines, G-CSF, and GM-CSF. This latter group can be subdivided into cytokines mediating humoral responses such as IL-4, IL-5, IL-6, IL-7, and IL-13, and those mediating cellular responses such as IL-1, IL-2, IL-3, IL-4, IL-7, IL-9, IL-10, IL-12, interferons (IFNs), IFN-g inducing factor (IGIF), transforming growth factor-beta (TNF-b), and tumor necrosis factor alpha and beta.

Beyond cytokines that circulate freely in blood, it has been discovered that CytoVesicles transport a wide range of biologically active cytokines that participate in concert with freely circulating cytokines to augment or promote acute and chronic pro-inflammatory disease conditions. The simultaneous reduction of circulating cytokines and CytoVesicles establishes a novel therapeutic strategy to alleviate the symptoms or severity of disease conditions associated with the abnormal production or dysregulation of pro-inflammatory cytokines.

An extracorporeal device for the broad-spectrum reduction of pro-inflammatory cytokines in blood and/or plasma is provided. As used herein, the extracorporeal device can be a plasma separation device. In a preferred embodiment, the extracorporeal device is an adsorptive toxin removal device, wherein blood or plasma is filtered through the device and inflammatory causing agents/toxins having diameters less than 0.60 microns can pass through pores and be bound, captured, and/or adsorbed by adsorbents in an extra-lumen space.

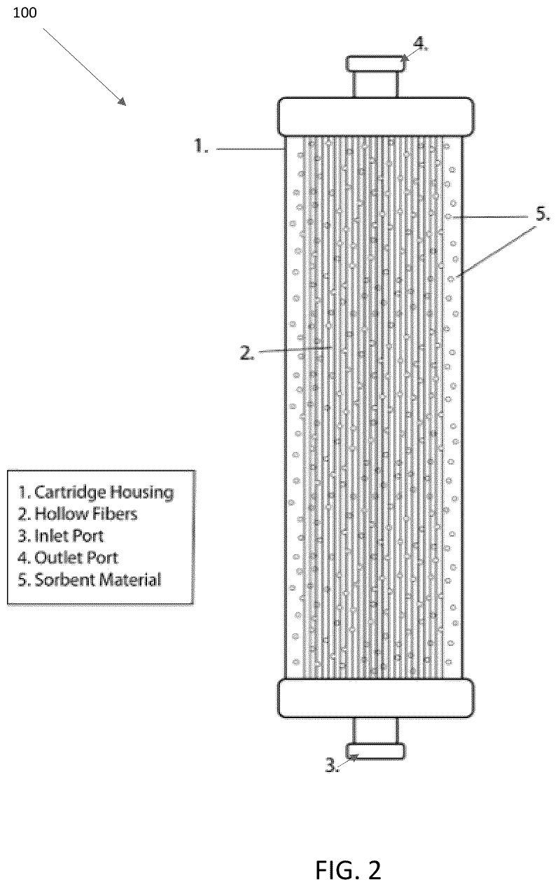

With reference to FIG. 2, the device 100 comprises a cartridge housing 1. As illustrated, housing 1 is transparent so as to reveal the internal components of the device 100. It will be appreciated, however, that the housing may be transparent, translucent, or opaque. Disposed within the housing 1 is a hollow fiber filter 2 comprised of a plurality of hollow fibers having fiber walls and a plurality of pores. The pores are sized and configured to allow inflammatory agents in blood or plasma as small as 0.5 nanometers and as large as 600 nanometers to pass through the walls of the hollow fibers. Agents and blood components with circulating cytokines, proteins, CytoVesicles, pathogens, endotoxins, exotoxins, and other targets with diameters less than 0.60 microns can thus pass through said plurality of pores into an extra-lumen space. By contrast, agents and blood components having diameters greater than about 0.60 microns are blocked by the fiber walls and cannot enter the extra-lumen space. The device 100 further includes an inlet port 3 for receiving unfiltered blood or plasma and an outlet port 4, wherein filtered blood or plasma exits the device for reintroduction into the circulatory system of the individual/patient.

Still with reference to FIG. 2, the extra-lumen space is populated with an adsorption component 5. An adsorption component 5, as used herein, refers to a substance which binds, captures, or otherwise adsorbs circulating inflammatory agents.

The adsorption component 5 can be activated carbon, non-ionic exchange resins, ion exchange resins, or combinations thereof. The activated carbon can include coated coconut shell granule, uncoated coconut shell granule, and/or synthetic charcoal. The activated carbon may have a pore size distribution of a micropore region of less than 100 Angstroms, a mesopore region of between about 100 and 1,000 Angstroms, and a macropore region of greater than 1,000 Angstroms. The non-ionic exchange resin can include non-ionic aliphatic ester resins, non-ionic polystyrene divinyl benzene resins, or any other suitable non-biologic adsorptive resin. In one aspect, the non-ionic aliphatic ester resin has an average surface area of approximately 500 m2/g, an average pore size of between about 300-600 Angstroms, and a mean particle diameter of about 560 microns. In another aspect, the non-ionic polystyrene divinyl benzene resin has an average surface area of approximately 700 m2/g, an average pore size of about 300 Angstroms, and a mean particle diameter from approximately 35 microns to approximately 120 microns. In still another embodiment, the non-ionic polystyrene divinyl benzene resin has an average surface area of approximately 600 m2/g, an average pore size of 100-400 Angstroms, and a mean particle diameter of between about 300 microns to about 500 microns. Adsorption component 5 can be applied to carriers which include, but are not limited, to coated or otherwise treated Alginate-Based Hydrogel Beads, perlite beads, Bio-Beads SM-2 Resin, and Bio-Beads S-X beads, or any suitable carrier as will be appreciated by a person of ordinary skill in the art,

The devices, systems and methods described herein support the broad-spectrum reduction of circulating cytokines and CytoVesicles from blood and blood plasma. To quantity cytokine and CytoVesicle reduction, assays and size exclusion techniques may be implemented to measure cytokine and CytoVesicle levels in blood and plasma prior to implementation of the devices, systems and methods described and then during and after completion of the devices, systems and methods described in the submission. Similarly, the reduction of toxins and pathogens can be quantified in blood or blood plasma.

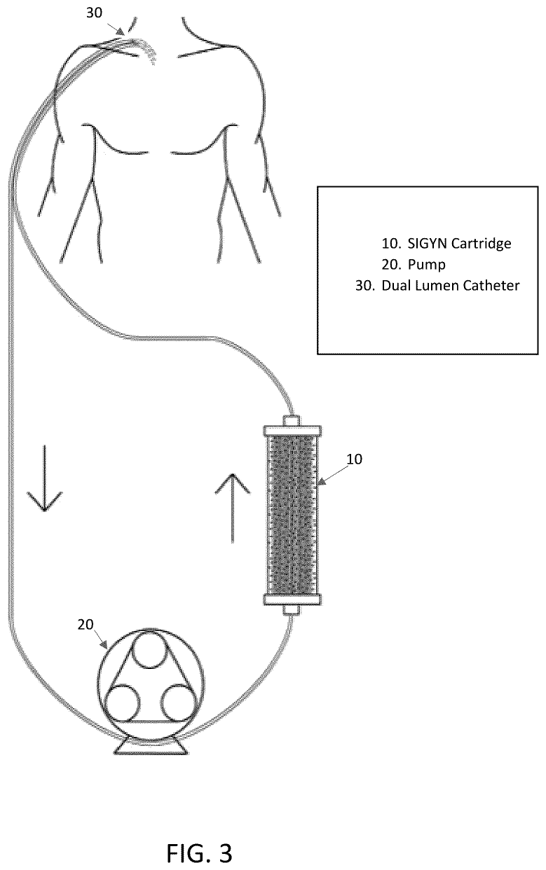

As illustrated in FIG. 3, disclosed herein is a methodology to reduce the systemic presence of inflammatory particles and is initiated through access to a patient's circulatory system. Access to the circulatory system can be obtained from arterial access or venous access. In one embodiment, access is obtained through the insertion of a central venous catheter into a patient. In a preferred embodiment, the catheter is a dual lumen catheter 30. Prior to initiation, a primary solution, which may include a saline or albumin solution is advantageously circulated throughout the device to improve hemocompatibility. Optionally, anticoagulant agents may be administered.

Once the device has been primed and access to the circulatory system established, the reduction or depletion of inflammatory particles from the blood or plasma occurs as an individual's blood or plasma passes through the extracorporeal device. As illustrated, the device 10 is configured to connect through the extracorporeal lines of the catheter to the patient's circulatory system. A pump 20 facilitates flow from the patient's circulatory system and through the extracorporeal device 10. The pump can be any approved device suitable for facilitating the extracorporeal filtration of blood and/or plasma. Exemplary pumps include dialysis pumps and CRRT machines. The device includes walls of porous hollow-fiber membranes, as described with reference to FIG. 2, wherein a formulation of adsorbent components are resident outside of the membrane walls and within the extra-lumen space between the outer shell of the cartridge and the hollow-fibers. The adsorbent components are formulated to bind, capture or adsorb a broad-spectrum of inflammatory particles that pass through the fiber walls to interact with the adsorbent components. As will be appreciated by a person of skill in the art, the blood or plasma is circulated to flow at rates sufficient to create pressure to cause plasma and inflammatory particles to flow through the fiber walls, but not at rates that would cause hemolysis. As blood or plasma is filtered through the device 10, the population of inflammatory particles is captured and reduced from the entire bloodstream, which is continuously infused back into the patient at rates equal to its removal during treatment.

Aspects of the invention are based upon the surprising discovery of a system and method for simultaneously removing circulating cytokines, CytoVesicles, cytokine aggregates, and endotoxins using a single extracorporeal device. Cytokine aggregates, as used herein, refer to two or more cytokines bound together. The removal of circulating cytokines, CytoVesicles, cytokine aggregates, and endotoxins in a single device without harming critical blood components creates considerable therapeutic benefits.

A method for treating a disease or disorder caused or exacerbated by a heightened inflammatory response is provided. The method includes providing an adsorptive toxin removal device such as the device described above with reference to FIG. 2. The device includes a housing, a hollow fiber plasma filter having a plurality of pores sized between about 200-1500 Angstroms, and a plurality of adsorbents positioned inside the housing and outside the hollow fiber filter. Plasma is filtered through the adsorptive toxin removal device such that inflammatory causing agents having a diameter less than 0.60 microns can pass through the pores of the hollow fiber filter and enter the extra-lumen space. The inflammatory causing agents are exposed to the plurality of adsorbents in the extra-lumen space such that the inflammatory agents are caused to be bound, captured, and/or adsorbed by the adsorbents, thereby reducing the amount of inflammatory causing agents in an individual's plasma.

As described above, the inflammatory causing agent can be a circulating cytokine. The circulating cytokine can be IL-1, TNF-a, IL-6, IL-11, IL-8 and other chemokines, G-CSF, and GM-CSF. IL-3, IL-4, IL-5, IL-6, IL-7, IL-9, IL-10, IL-13 and transforming growth factor-b (TGF-b), and combinations thereof, as well as those contributing to cellular inflammation such as IL-1, IL-2, IL-3, IL-4, IL-7, IL-9, IL-10, IL-12, interferons (IFNs), IFN-g inducing factor (IGIF), TGF-b, and TNF-a and -b.

The removal or reduction in the number of inflammatory causing agents in the blood or plasma is useful for the treatment of a wide range of diseases or disorders caused or exacerbated by an abnormal production or dysregulation of pro-inflammatory cytokines. These diseases or conditions can include, without limitation, conditions associated with cytokine storm syndrome (CSS), virus induced cytokine storm, bacteria-induced cytokine storm, acute respiratory distress syndrome (ARDS), cytokine release syndrome (CRS), graft-versus-host disease (GVHD), sepsis, systemic inflammatory response syndrome (SIRS), hepatic encephalopathy, acute kidney injury (AKI), severe pneumonia, and combinations thereof.

In addition to treating diseases and disorders associated with abnormal production or dysregulation of pro-inflammatory cytokines, devices and methods described herein are useful for capturing biologic toxins such as endotoxins and exotoxins shed or released by bacteria. The bacteria can be gram negative or gram positive.

The reduction in pro-inflammatory cytokines, CytoVesicles, cytokine aggregates, and/or endotoxins can also improve outcomes with respect to organ transplantation. The reduction and/or elimination of circulating inflammatory articles increases viability of donor organs for transplant. Moreover, the filtration of donor blood to remove pro-inflammatory articles, endotoxin, exotoxin, and the like can purify donor blood prior to infusion into a human recipient.

Additionally, a method for reducing the presence of unwanted drug agents from the bloodstream is provided. The method includes providing an extracorporeal device having a hollow fiber plasma filter and a plurality of adsorbents positioned inside the housing and outside the hollow fibers, filtering blood or plasma through the hollow fiber plasma filter, allowing the unwanted drug agent or agents to pass through the pores of the hollow fiber and into the extra-lumen space, and adsorbing, capturing, or otherwise binding the drug agents to an adsorbent as described herein. The drug agent can be any pharmaceutical agent, illicit drug, controlled substance, or similar compound for which the presence of said drug agent at above a certain threshold level in the blood causes a deleterious effect. Drugs can include, without limitation, acetaminophen, barbiturates, narcotics, and chemotherapeutic agents.

To quantify the reduction of inflammatory particles from the blood or plasma, assays, size exclusion techniques, and image measurement technologies may be utilized to measure levels of inflammatory particles in patient blood or plasma from a sample of patient blood or plasma taken before and after administration of the therapy.

What is claimed is:

1. An extracorporeal system for the removal of inflammatory agents from blood, comprising:
a housing;
a hollow fiber filter disposed within said housing; said filter comprising a plurality of pores sized between about 200-6000 Angstroms and configured to permit passage of inflammatory agents having a diameter less than 0.60 microns from within the filter to an extra lumen space disposed within the housing and outside the filter; and
at least one adsorption component positioned inside the housing and outside the hollow fiber in an extra-lumen space;
wherein at least one adsorption component comprises a non-ionic exchange resin selected from the group consisting of non-aliphatic ester resins, non-ionic polystyrene divinyl benzene resins, and other non-biologic adsorptive resins; and
wherein the inflammatory agents are selected from the group consisting of cytokines, proteins with surface-bound cytokines, cytovesicles with encapsulated cytokine cargos, cytovesicles with surface-bound cytokines, pathogens, bacterial endotoxins, and bacterial exotoxins.

2. The extracorporeal system of claim 1, wherein the at least one adsorption component further comprises at least one of activated carbon and ion exchange resin.

3. The extracorporeal system of claim 1, wherein said plurality of pores are sized and dimensioned to prevent a blood agent having a diameter greater than 0.60 microns to pass through the fiber walls and interact with adsorption components in the extra-lumen space.

4. The extracorporeal system of claim 2, wherein said activated carbon comprises coated or uncoated coconut shell granule or synthetic charcoal.

5. The extracorporeal system of claim 1, wherein at least one of said non-ionic aliphatic ester resins has an average surface area of approximately 500 m2/g, an average pore size of approximately 300-600 angstroms, and a mean particle diameter of 560 microns.

6. The extracorporeal system of claim 1, wherein at least one of said non-ionic polystyrene divinyl benzene resins has an average surface area of approximately 500 m2/g, and an average pore size of approximately 300-600 angstroms, and a mean particle diameter of 560 microns.

7. The extracorporeal system of claim 1, wherein at least one of said non-ionic polystyrene divinyl benzene resins has an average surface area of approximately 700 m2/g, an average pore size of 300 Angstroms, and a mean particle diameter from approximately 35 microns to approximately 120 microns.

8. The extracorporeal system of claim 1, wherein at least one of said non-ionic polystyrene divinyl benzene resins has an average surface area of approximately 600 m2/g, an average pore size of 100-400 Angstroms, and a mean particle diameter from approximately 300 microns to approximately 500 microns.

9. The extracorporeal system of claim 2, wherein the activated carbon has a pore size distribution of a Micropore region of less than 100 Angstroms, a Mesopore region of between 100 and 1,000 Angstroms, and a Macropore region of greater than 1,000 Angstroms.

10. A method for treating a disease or disorder in an individual in need thereof, comprising:
providing an extracorporeal adsorptive toxin removal device, said device having:
a housing;
a hollow fiber plasma filter having a plurality of pores sized between 200-6000 Angstrom disposed within the housing; and
an adsorbent positioned inside the housing and outside the fiber filter in the extra lumen space;
filtering the plasma of an individual in need thereof through said adsorptive toxin removal device; wherein said filtering causes an inflammatory causing agent with a diameter less than 0.60 microns to pass through said pores;
contacting said inflammatory causing agent with said absorbent; wherein said inflammatory causing agent binds to said adsorbent; and
capturing said inflammatory causing agent in said adsorbent;
wherein the adsorbent comprises a non-ionic exchange resin selected from the group consisting of non-ionic aliphatic ester resins, non-ionic polystyrene divinyl benzene resins, and other non-biologic adsorptive resins; and
wherein the inflammatory agents are selected from the group consisting of cytokines, proteins with surface bound cytokines, cytovesicles with encapsulated cytokine cargos, cytovesicles with surface bound cytokines, pathogens, bacterial endotoxins, and bacterial exotoxins.

11. The method of claim 10, wherein the capture of said inflammatory causing agent prevents said agent from reentering circulation.

12. The method of claim 10, wherein said cytokines are selected from the group consisting of: IL-1, TNF-a, IL-11, IL-8, G-CSF, and GM-CSF. IL-3, IL-5, IL-7, IL-9, and transforming growth factor-b (TGF-b).

13. The method of claim 10, wherein said cytokines are selected from the group consisting of IL-2, IL-3, IL-4, IL-6, IL-10, IL-12, IL-13, interferons (IFNs), IFN-g inducing factor (IGIF), and TNF-a and -b.

14. The method of claim 10, wherein said disease or disorder is selected from the group consisting of cytokine storm syndrome (CSS), virus induced cytokine storm, bacteria induced cytokine storm, acute respiratory distress syndrome (ARDS), cytokine release syndrome (CRS), graft-versus-host disease (GVHD), sepsis, systemic inflammatory response syndrome (SIRS), hepatic encephalopathy, acute kidney injury (AKI) and pneumonia.

15. The method of claim 10, wherein said inflammatory agent is a biologic toxin.

16. The method of claim 15, wherein said toxin is a bacterial endotoxin or exotoxin.

17. A method of simultaneously removing circulating cytokines, CytoVesicles, cytokine aggregates, and bacterial endotoxins from the blood of an individual in need thereof; comprising:
accessing an extracorporeal line to an individual's circulatory system with a catheter;
providing the extracorporeal system, wherein said system has an inlet port and an outlet port;
connecting said line to the inlet port;
attaching a second extracorporeal line to said outlet port;
controlling the flow of blood through said extracorporeal system with a pump;
filtering the blood through the hollow fibers of said extracorporeal system; wherein said filtering causes said circulating cytokines, CytoVesicles, cytokine aggregates, and bacterial endotoxins to pass through the pores and into said extra-lumen space; and
contacting said filtered circulating cytokines, CytoVesicles, cytokine aggregates, and bacterial endotoxins with said adsorbent; wherein said adsorbent captures said cytokines, CytoVesicles, cytokine aggregates, and endotoxins.
